(19) United States
(12) Patent Application Publication
(10) Pub. No.: US 2024/0185417 A1
(43) Pub. Date: Jun. 6, 2024

(54) SYSTEMS AND METHODS FOR PROCESSING MAMMOGRAPHY IMAGES

(71) Applicant: United Imaging Intelligence (Beijing) Co., Ltd., Beijing (CN)

(72) Inventors: Zhang Chen, Brookline, MA (US); Shanhui Sun, Lexington, MA (US); Xiao Chen, Lexington, MA (US); Yikang Liu, Cambridge, MA (US); Terrence Chen, Lexington, MA (US); Xiangyi Yan, Irvine, CA (US)

(73) Assignee: United Imaging Intelligence (Beijing) Co., Ltd., Beijing (CN)

(21) Appl. No.: 18/074,297

(22) Filed: Dec. 2, 2022

(51) Int. Cl. G06T 7/00; G06T 7/11

(52) U.S. Cl. CPC G06T 7/0014; G06T 7/11; G06T 2207/20084; G06T 2207/30068

(57) ABSTRACT

Mammography may provide multi-view information about the healthy state of a person's breasts, and described herein are deep learning based techniques for obtaining mammographic images associated with multiple views, extracting cross-view features from the images, and automatically determining the existence or non-existence of a medical abnormality based on the extracted features. The cross-view features may be determined using an encoding module of an artificial neural network (ANN) and the encoded features may be decoded using a decoding module of the ANN to generate a prediction (e.g., a classification label, a bounding shape, a segmentation mask, a probability map, etc.) about the medical abnormality.

### BACKGROUND

Breast cancer accounts for a large portion of new cancer cases and hundreds of thousands of deaths each year. Early screening and detection are key to improving the outcome of breast cancer treatment, and can be accomplished through mammography exams (mammograms). While mammographic images taken during a mammogram may provide multi-view information about the healthy state of the breasts, conventional image processing techniques may not be capable of extracting and fusing the information encompassed in the multiple views, thus preventing mammography from reaching its full potential.

### SUMMARY

Described herein are systems, methods, and instrumentalities associated with processing mammographic images including digital breast tomosynthesis (DBT) images and full-field digital mammography (FFDM) images. An apparatus capable of performing such processing tasks may include at least one processor configured to obtain one or more first medical images of a person and one or more second images of the person, wherein the one or more first images may be associated with a first mammographic view of the person and the one or more second medical images may be associated with a second mammographic view of the person. The at least one processor may be further configured to determine a first set of features associated with the one or more first medical images and a second set of features from the one or more second medical images, and to derive a third set of features based on the first set of features and the second set of features, wherein the third set of features may represent mammographic characteristics of the person indicated by the first mammographic view and the second mammographic view. Based at least on the third set of features, the at least one processor may be configured to predict the existence or non-existence of a medical abnormality, for example, by generating an output image that may include an indication (e.g., a bounding shape, a segmentation mask, a heat map, etc.) of the medical abnormality or a classification label that may indicate the existence or non-existence of the medical abnormality.

In examples, the at least one processor described herein may be configured to generate a first output image that includes a first indication of the medical abnormality and a second output image that includes a second indication of the medical abnormality, wherein the first output image may correspond to the first mammographic view and the second output image may correspond to the second mammographic view. In examples, the third set of features described herein may be determined using an artificial neural network (ANN), which may include a first encoding module and/or a second encoding module. The first encoding module may be configured to determine the first set of features associated with the one or more first medical images or the second set of features associated with the one or more second medical images, while the second encoding module may be configured to determine the third set of features. In examples, the ANN may further include a decoding module configured to predict the existence or non-existence of the medical abnormality based at least on the third set of features.

### BRIEF DESCRIPTION OF THE DRAWINGS

A more detailed understanding of the examples disclosed herein may be gained from the following description, given by way of example in conjunction with the accompanying drawings.

FIG. 1A and FIG. 1B are simplified diagrams illustrating examples of mammography techniques, according to some embodiments described herein.

FIG. 2 is a simplified diagram illustrating an example of a machine-learning (ML) framework for processing mammographic images such as DBT images or FFDM images, according to some embodiments described herein.

FIG. 3 is a simplified diagram illustrating an example architecture of an ML framework for processing mammographic images, according to some embodiments described herein.

FIG. 4 is a flow diagram illustrating an example method for processing mammographic images, according to some embodiments described herein.

FIG. 5 is a flow diagram illustrating an example method for training a neural network to perform one or more of the tasks as described with respect to some embodiments provided herein.

FIG. 6 is a simplified block diagram illustrating an example system or apparatus for performing one or more of the tasks as described with respect to some embodiments provided herein.

### DETAILED DESCRIPTION

The present disclosure is illustrated by way of example, and not by way of limitation, in the figures of the accompanying drawings. A detailed description of illustrative embodiments will now be described with reference to the various figures. Although this description provides a detailed example of possible implementations, it should be noted that the details are intended to be exemplary and in no way limit the scope of the application.

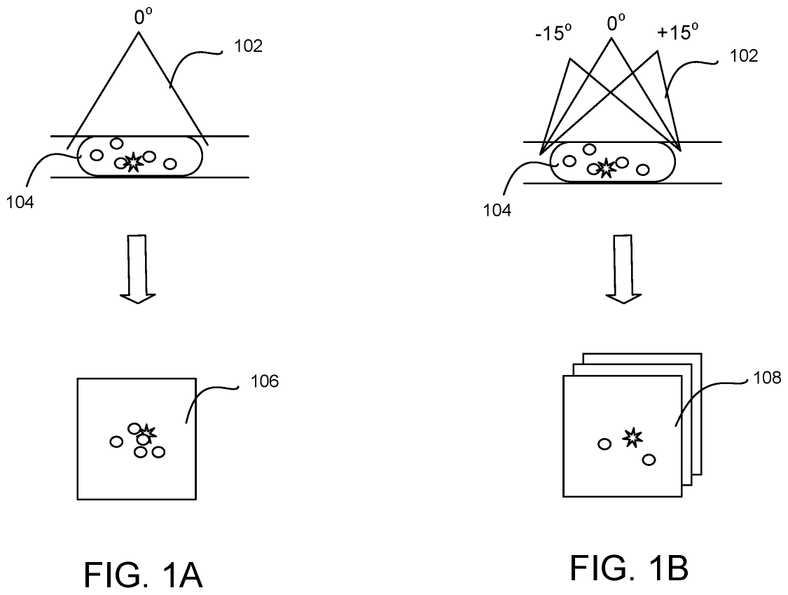

Mammography (mammogram) may be used to capture pictures of a breast from different views (e.g., a craniocaudal (CC) view and/or a mediolateral oblique (MLO) view). A standard mammogram may include images of multiple mammographic views including, e.g., a left CC (LCC) view, a left MLO (LMLO) view, a right CC (RCC) view, and a right MLO (RMLO) view. FIGS. 1A and 1B illustrate examples of mammogram techniques, with FIG. 1A showing an example of full-field digital mammography (FFDM) and FIG. 1B showing an example of digital breast tomosynthesis (DBT). As illustrated by FIG. 1A, FFDM may be considered a 2D imaging modality and may involve passing a burst of X-rays 102 through a compressed breast 104 at a certain angel (e.g., perpendicular to the breast), capturing the X-rays 102 on the opposite side (e.g., using a solid-state detector), and producing a 2D image 106 of the breast based on the captured signals (e.g., the captured X-rays 102 may be converted to electronic signals, which may then be used to generate the 2D image 106). Using FFDM, the information of the whole breast may be incorporated into a single 2D image (e.g., the 2D image 106), with an increased chance that normal breast tissues (e.g., represented by circles in FIG. 1A) and a potential lesion (e.g., represented by a star in FIG. 1A) may overlap in the resulting mammogram image. Such an overlap, if occurs, may obscure the lesion and increase the chance of false positive or false negative diagnoses.

The DBT technology illustrated by FIG. 1B may achieve or resemble the quality of a 3D imaging modality (e.g., DBT may be considered a pseudo 3D imaging modality). As shown, the technology may involve passing the burst of X-rays 102 through the compressed breast 104 at different angles (e.g., 0°, +15°, −15°, etc.) during a scan, acquiring a set of X-ray images of the breast at each of those angles, and reconstructing the X-ray images acquired at the different angles into a series of slices 108 (e.g., one slice for each angle) that may then be displayed individually or as a movie (e.g., in a dynamic cine mode).

Both the FFDM technique shown in FIG. 1A and the DBT techniques shown in FIG. 1B may be applied to acquire medical images of the breasts that may represent different views of the breasts (e.g., the RCC, LCC, LMLO, and RMLO views described herein). Information provided by the different views may then be used to distinguish normal breast tissues (e.g., represented by circles in FIGS. 1A and 1B) from a potential lesion (e.g., represented by a star in FIGS. 1A and 1B) and achieve improved diagnostic and screening results. It should be noted that although FIG. 1B shows only three angles at which x-ray images of the breast 104 are taken, those skilled in the art will appreciate that more angles may be used and more images may be taken during a DBT procedure. For example, 15 images of the breast may be taken along an arc from the top and side of the breast, and the images may be reconstructed into multiple non-overlapping slices. Those skilled in the art will also appreciate that, although not shown in FIGS. 1A and 1B, a FFDM or DBT scan may generate different views of both breasts including, for example, the LCC, LMLO, RCC, and/or RMLO views described herein.

While the mammogram techniques described herein may provide rich information about potential breast diseases (e.g., such as breast cancer), the amount of data generated during a mammogram procedure (e.g., 40 to 80 slices per view per breast during a DBT procedure) may be large and difficult to analyze based only on human efforts (e.g., manually by a radiologist). Hence, in embodiments of the present disclosure, artificial intelligence (AI) or deep-learning (DL) based techniques are employed to automatically dissect, analyze, and/or present mammogram data (e.g., FFDM and/or DBT data) in ways that take advantage of the information encompassed in multiple views of the breasts to gain more holistic and accurate insights into the health conditions of the breasts. It should be noted that the terms “artificial intelligence model,” “deep learning model,” “machine learning (ML) model,” and “artificial neural network” may be used interchangeably herein, and the term “mammogram images” or “mammographic images” may include FFDM images or DDT images.

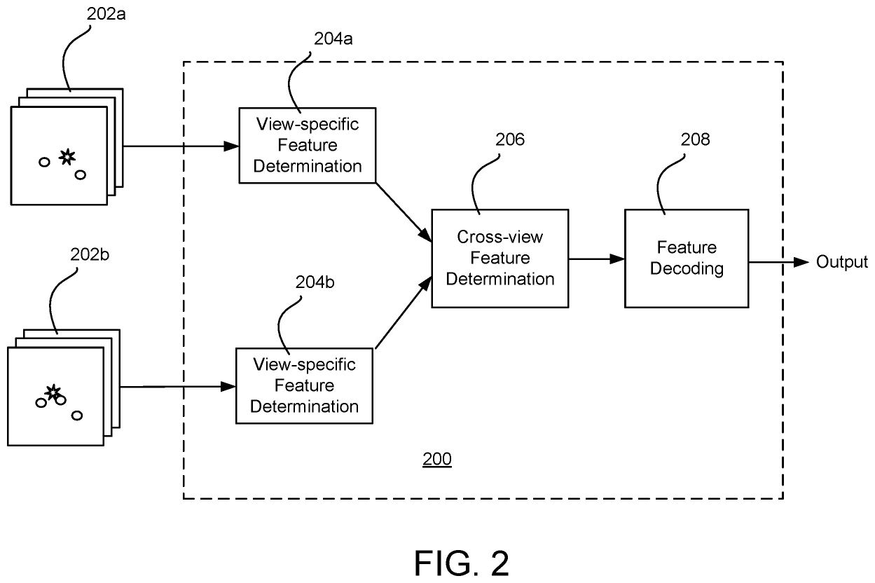

FIG. 2 illustrates an example of an ML framework 200 for processing medical images (e.g., mammogram images) of a breast. As shown, the medical images may be associated with multiple mammographic views of a person such as a first view 202a and a second view 202b. While two mammographic views are shown in FIG. 2, those skilled in the art will appreciate that the techniques disclosed herein may also be used to process a single mammographic view (e.g., corresponding to a single input) or more than two mammographic views (e.g., corresponding to multiple inputs). As described herein, each mammographic view 202a or 202b may correspond to a respective set of images (e.g., one or more first images corresponding to the first view 202a and one or more second images corresponding to the second view 202b). The ML framework 200 may be configured to determine (e.g., extract) the features of the images associated with the first and second views 202a and 202b (e.g., from each individual image associated with view 202a or 202b), fuse and/or encode the extracted features (e.g., into one or more single-view feature representations and/or one or more cross-view feature representations), and generate an output that may indicate the existence or non-existence of a medical abnormality (e.g., a lesion) in a breast, for example, by decoding the encoded feature representations. For example, image features associated with the first view 202a and second view 202b may be determined at 204a and 204b, respectively, and these view-specific features (e.g., single-view features) may be used to determine a third set of features (e.g., cross-view features) at 206 that may represent the mammographic characteristics of the person as indicated by the first view 202a and second view 202b. The cross-view features may then decoded at 208 to generate the output described above. In examples, such an output may include a classification label indicating (e.g., via a Boolean value or on a scale of 0 to 1) whether a medical abnormality such as a lesion is identified in the mammogram images. In examples, such an output may include one or more images with respective bounding shapes (e.g., bounding boxes) drawn around the medical abnormality to indicate the location of the medical abnormality. In examples, such an output may include one or more segmentation masks (e.g., binary images) or one or more probability maps (e.g., heat maps comprising probability values) in which pixels of a first value (or in a first range) may indicate that those pixels belong to an abnormal area and pixels of a second value (or in a second range) may indicate that those pixels do not belong to the abnormal area.

In examples, a convolutional neural network (CNN) may be trained to extract features from the input mammogram images. Such a CNN may include a plurality of layers such as one or more convolution layers, one or more pooling layers, and/or one or more fully connected layers. Each of the convolution layers may include a plurality of convolution kernels or filters configured to extract features from an input image (e.g., a FFDM or DBT image) received through an input channel. The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map).

In examples, an artificial neural network (ANN) (e.g., including the CNN described above or separate from the CNN described above) may be trained to encode and/or decode the cross-view features described herein. For instance, such an ANN may include a first encoding module, a second encoding modules, and/or a decoding module. The first encoding module may be trained to determine the view-specific features described herein (e.g., based on local features of the input images), while the second encoding module may be trained to generate the cross-view feature representation (e.g., comprising global features) described herein (e.g., based on local features extracted by the CNN from the input images and/or view-specific features fused from those local features). The decoding module, on the other hand, may be trained to decode the features determined by the first encode module (e.g., the view-specific features) and/or the second encoding module (e.g., the cross-view features), and to generate the output described herein based on the decoded features.

In examples, the decoding module may include one or more un-pooling layers and one or more transposed convolution layers that may be configured to up-sample and de-convolve the feature representations generated by the encoding modules. As a result of the up-sampling and de-convolution, a dense feature representation (e.g., a dense feature map) of the input images may be derived and a prediction about the existence or non-existence of a medical abnormality in the breasts may be made based on the dense feature representation. In examples, the decoding module may be trained to acquire object query capabilities and rely on such capabilities to identify features determined by the encoding module(s) (e.g., which may be stored in a feature memory or feature database) that may be associated with a medical abnormality.

In examples, the ANN described herein may adopt a transformer neural network architecture under which one or more self-attention modules may be used to determine the view-specific (e.g., single-view) features described, one or more cross-attention modules may be used to determine the cross-view features described herein, and one or more decoding modules may be used to decode the encoded features. The self-attention and/or cross-attention modules (e.g., each of the attention modules) may include a stack of layers (e.g., six identical layers) and each layer may include a set of sub-layers (e.g., a multi-head self-attention mechanism and a position-wise fully connected feed-forward network). A residual connection may be employed around each of the sub-layers, followed by layer normalization. This way, the output of each sub-layer may be LayerNorm(x+Sublayer(x)), where Sublayer(x) may be a function implemented by the sub-layer itself. The decoding module may also include a stack of layers (e.g., six identical layers) and a set of sub-layers (e.g., two sub-layers as described above and a third sub-layer configured to perform multi-head attention over the output of the encoder stack). Residual connections may be employed around each of the sub-layers, followed by layer normalization. The sub-layer in the decoding module stack may be configured to prevent positions from attending to subsequent positions. Such masking, combined with offsetting the output embeddings by one position, may ensure that the predictions for position i may depend (e.g., only depend) on the known outputs at positions less than i. The attention mechanism described herein may map a query and a set of key-value pairs to an output (e.g., the query, keys, values, and output may be vectors), and the output may be computed as a weighted sum of the values, where the weight assigned to each value may be computed by a compatibility function of the query with the corresponding key. The fully connected feed-forward network described herein may be applied to each position separately and/or identically (e.g., by performing two linear transformations with a ReLU activation in between).

Using the network architecture and techniques described herein, information encompassed in view 202a and view 202b may be fused together (e.g., the transformer may model the relationship among different regions or different views of the breasts) and insight gained from one view (e.g., view 202a) may be supplemented by insights from another view (e.g., view 202b) such that a medical abnormality may be predicted with improved accuracy. It should be noted that while the operations at 204a/204b, 206, and 208 may be shown in FIG. 2 as being separate from each other (e.g., accomplished using separate neural networks), those skilled in the art will appreciated that the functionalities may also be accomplished using one neural network, for example, with a CNN backbone for extracting local features from the input images and respective sub-networks or modules (e.g., encoding and/or decoding modules) for implementing the other functionalities.

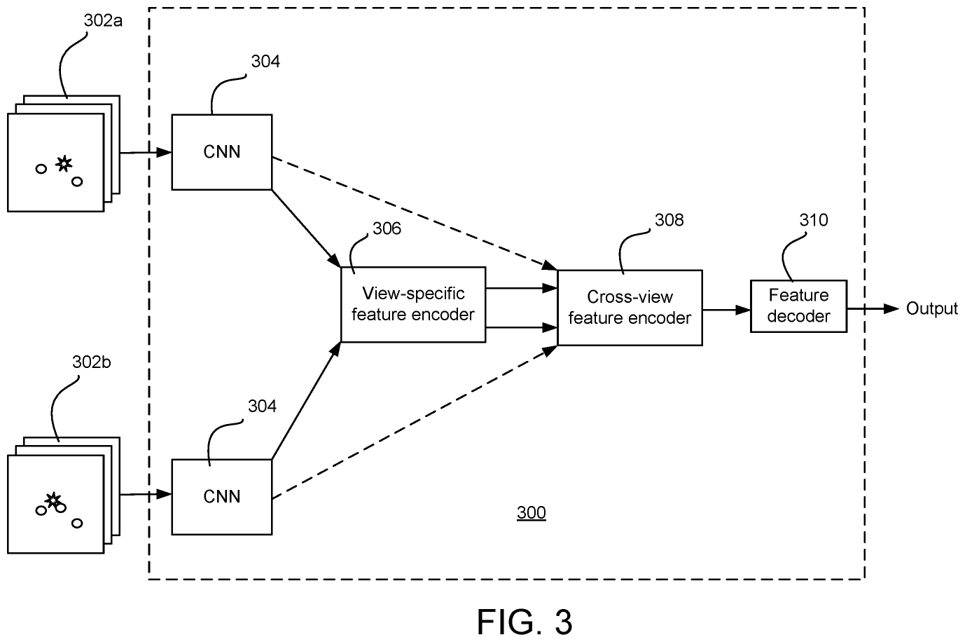

FIG. 3 illustrates an example of an ML framework 300 (e.g., the ML framework 200 of FIG. 2) for processing mammographic images (e.g., DBT and/or FFDM images). Similar to FIG. 2, even though two views (e.g., 302a and 302b) are shown in FIG. 3, those skilled in the art will appreciate that the ML framework 300 may be capable of processing just one view (e.g., as a single input) or more than two views (e.g., as multiple inputs). As described herein, each view 302a or 302b may be associated with one or more images, and ML framework 300 may include a CNN 304 for extracting features (e.g., local features) from the image(s) associated with view 302a or 302b and representing the extracted features with respective features maps or feature vectors (e.g., one feature map per image). The ML framework 300 may further include a first encoding module 306 (e.g., a self-attention or view-specific encoder) trained to fuse the features extracted by CNN 304 into respective view-specific features representing the mammographic characteristics of each view. The view-specific (e.g., single-view) feature representations obtained via first encoding module 306 may be stored (e.g., in respective memory areas) and provided to other modules or components of the ML framework to facilitate the prediction of a medical abnormality (e.g., a lesion) in the breast.

The ML framework 300 may additionally include a second encoding module 308 (e.g., a cross-attention or cross-view encoder) trained to determine cross-view features of the breasts based on the view-specific features fused by first encoding module 306 and/or the features extracted by CNN 304. These cross-view features may be stored in a cross-view feature memory, from which (e.g., together with the single-view features) a feature decoder 310 of the ML framework 300 may query (e.g., identify) features associated with a medical abnormality and generate an output indicating the existence or non-existence of the medical abnormality. As described herein, such an output may include a classification label indicating whether the medical abnormality is identified in the input images. The output may also include an image (e.g., a main view of the breast) with a bounding shape (e.g., bounding box) drawn around the medical abnormality or multiple images (e.g., representing multiple views of the breast) with respective bounding shapes drawn around the medical abnormality. The image(s) generated by decoder 310 may also include a segmentation mask (e.g., a binary image) for the medical abnormality, a heat map (e.g., with color-coded probability values) associated with the medical abnormality, etc.

As explained herein, view 302a or 302b may include an LCC view, an RCC view, an LMLO view, an RMLO view, etc. ML framework 300 may operate to generate a single output (e.g., a main view of the breast(s) with a bounding shape drawn around the medical abnormality) based on a single input (e.g., one or more images associated with a single view) or multiple inputs (e.g., multiple views). ML framework 300 may operate to generate multiple outputs (e.g., multiple views of the breasts with respective bounding shapes drawn around the medical abnormality) based on multiple inputs (e.g., multiple views). Also as explained herein, feature encoder 306 may be trained to fuse the features associated with a specific view, while feature encoder 308 may be trained to fuse the features associated with multiple views. As such, the operations of these encoders may have the effect of extracting features from the input images in a global fashion. By integrating and learning from these global features, ML framework 300 may be able to make predictions that are more accurate and robust than those made by treating each mammographic view in isolation.

In some examples, feature decoder 310 may generate prediction results based only based on the cross-view features determined by feature encoder 308. In other examples, feature decoder 310 may generate the prediction results based on the cross-view features and the view-specific features determined by feature encoder 306. In either case, the cross-view features may be obtained by utilizing the information encompassed in the view-specific features (e.g., even if only the cross-view features are used by feature decoder 310 to generate a prediction). In examples, ML learning framework 300 may take images associated with multiple views as inputs, but may make predictions for only a subset of the views. In examples, ML framework 300 may take images associated with a specific view as an input and make a prediction for that view (e.g., in a single-input single-output scenario). In these examples, feature encoder 308 may not be needed and the global features of the breasts may be determined by feature decoder 310 based on view-specific features provided by feature encoder 306.

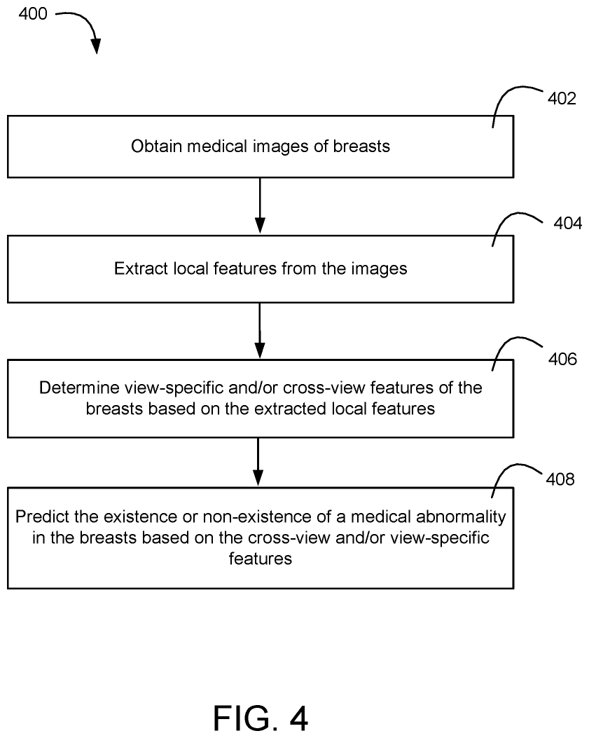

FIG. 4 illustrates an example method 400 for processing mammographic images (e.g., DBT or FFDM images). The method 400 may be implemented by an ML-based system or apparatus as described herein, and may include obtaining images associated with multiple views of a person's breasts at 402. The method 400 may further include extracting (e.g., using a CNN backbone) respective features (e.g., local features) from the images at 404 and determine, at 406, view-specific features (e.g., global features of each view) and/or cross-view features of the breasts based on the features extracted at 404. The method may additionally include decoding, at 408, the cross-view and/or view-specific features of the breasts and making a prediction about the existence or non-existence of a medical abnormality in the breasts based on the decoding.

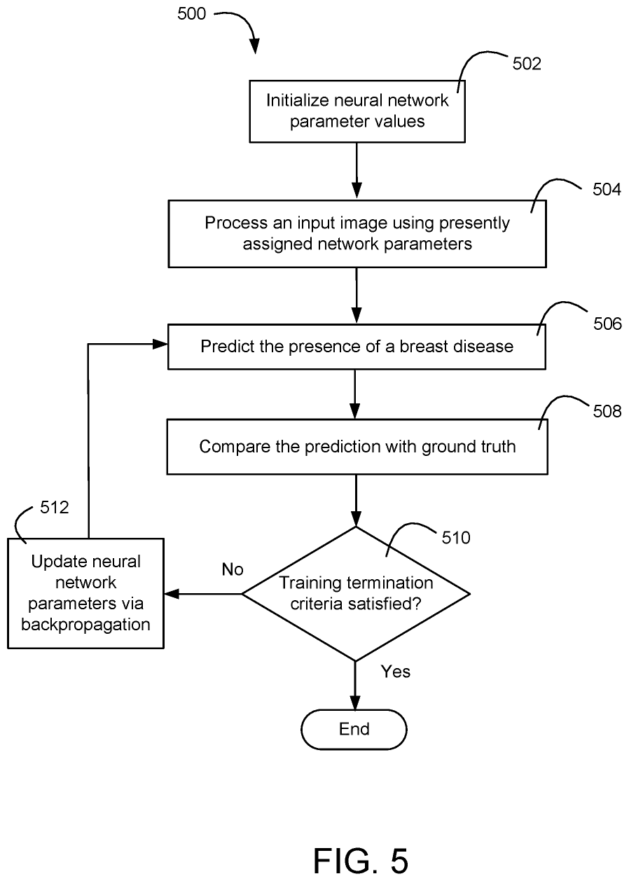

FIG. 5 shows a flow diagram illustrating an example process 500 for training a neural network (e.g., a neural network included in ML framework 200 of FIG. 2 or ML framework 300 of FIG. 3) to perform one or more of the tasks described herein. As shown, the training process 500 may include initializing the operating parameters of the neural network (e.g., weights associated with various layers of the neural network) at 502, for example, by sampling from a probability distribution or by copying the parameters of another neural network having a similar structure. The training process 500 may further include processing one or more input images (e.g., one or more mammogram images) using presently assigned parameters of the neural network at 504, and making a prediction about the presence of a medical abnormality (e.g., a breast disease such as a lesion) in the input images at 506. The prediction result may be compared, at 508, to a ground truth that indicates a true status of the medical abnormality (e.g., whether the breast disease truly exists) to determine a loss associated with the prediction based on a loss function. The loss function employed for the training may be selected based on the specific task that the neural network is trained to do. For example, if the task involves a classification or segmentation of the input image, a loss function based on calculating a mean squared error between the prediction result and the ground truth, or based on determining an L1 or L2 norm, etc., may be used. If the task involves detecting the location of an abnormal area (e.g., by drawing a bounding box) around the abnormal area, a loss function based on generalized intersection over union (GIOU) may be used.

At 510, the loss calculated using one or more of the techniques described above may be used to determine whether one or more training termination criteria are satisfied. For example, the training termination criteria may be determined to be satisfied if the loss is below a threshold value or if the change in the loss between two training iterations falls below a threshold value. If the determination at 510 is that the termination criteria are satisfied, the training may end; otherwise, the presently assigned network parameters may be adjusted at 512, for example, by backpropagating a gradient descent of the loss function through the network before the training returns to 506.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed.

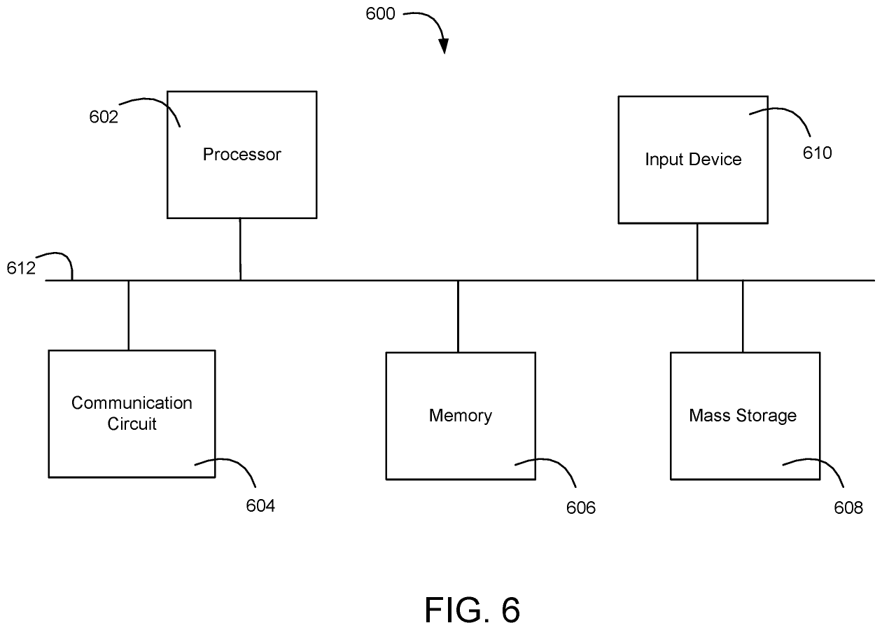

The systems, methods, and/or instrumentalities described herein may be implemented using one or more processors, one or more storage devices, and/or other suitable accessory devices such as display devices, communication devices, input/output devices, etc. FIG. 6 illustrates an example apparatus 600 that may be configured to perform the tasks described herein. As shown, apparatus 600 may include a processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked or clustered) with other computation devices to perform the tasks described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
at least one processor configured to:
obtain one or more first medical images of a person and one or more second medical images of the person, wherein the one or more first medical images are associated with a first mammographic view of the person and wherein the one or more second medical images are associated with a second mammographic view of the person;
determine a first set of features associated with the one or more first medical images;
determine a second set of features associated with the one or more second medical images;
determine a third set of features based on the first set of features and the second set of features, wherein the third set of features represents mammographic characteristics of the person indicated by the first mammographic view and the second mammographic view; and
predict the existence or non-existence of a medical abnormality based at least on the third set of features.

2. The apparatus of claim 1, wherein the at least one processor being configured to predict the existence or non-existence of the medical abnormality comprises the at least one processor being configured to generate a first output image that includes a first indication of the medical abnormality.

3. The apparatus of claim 2, wherein the first indication includes a bounding shape around the medical abnormality.

4. The apparatus of claim 2, wherein the first indication includes a segmentation mask or a heat map associated with the medical abnormality.

5. The apparatus of claim 2, wherein the at least one processor being configured to predict the existence or non-existence of the medical abnormality further comprises the at least one processor being configured to generate a second output image that includes a second indication of the medical abnormality, the first output image corresponding to the first mammographic view, the second output image corresponding to the second mammographic view.

6. The apparatus of claim 1, wherein the at least one processor being configured to predict the existence or non-existence of the medical abnormality comprises the at least one processor being configured to generate a classification label indicating the existence or non-existence of the medical abnormality.

7. The apparatus of claim 1, wherein the one or more first medical images include a first digital breast tomosynthesis (DBT) image or a first full-field digital mammography (FFDM) image, and wherein the one or more second medical images include a second DBT image or a second FFDM image.

8. The apparatus of claim 1, wherein the third set of features is determined using an artificial neural network (ANN).

9. The apparatus of claim 8, wherein the ANN includes first encoding module configured to determine the first set of features associated with the one or more first medical images or the second set of features associated with the one or more second medical images, the ANN further including a second encoding module configured to determine the third set of features.

10. The apparatus of claim 9, wherein the ANN further includes a decoding module configured to predict the existence or non-existence of the medical abnormality based at least on the third set of features.

11. A method of processing medical images, the method comprising:
obtaining one or more first medical images of a person and one or more second medical images of the person, wherein the one or more first medical images are associated with a first mammographic view of the person and wherein the one or more second medical images are associated with a second mammographic view of the person;
determining a first set of features associated with the one or more first medical images;
determining a second set of features associated with the one or more second medical images;
determining a third set of features based on the first set of features and the second set of features, wherein the third set of features represents mammographic characteristics of the person indicated by the first mammographic view and the second mammographic view; and
predicting the existence or non-existence of a medical abnormality based at least on the third set of features.

12. The method of claim 11, wherein predicting the existence or non-existence of the medical abnormality comprises generating a first output image that includes a first indication of the medical abnormality.

13. The method of claim 12, wherein the first indication includes a bounding shape around the medical abnormality.

14. The method of claim 12, wherein the first indication includes a segmentation mask or a heat map associated with the medical abnormality.

15. The method of claim 12, wherein predicting the existence or non-existence of the medical abnormality further comprises generating a second output image that includes a second indication of the medical abnormality, the first output image corresponding to the first mammographic view, the second output image corresponding to the second mammographic view.

16. The method of claim 11, wherein predicting the existence or non-existence of the medical abnormality comprises generating a classification label indicating the existence or non-existence of the medical abnormality.

17. The method of claim 11, wherein the one or more first medical images include a first digital breast tomosynthesis (DBT) image or a first full-field digital mammography (FFDM) image, and wherein the one or more second medical images include a second DBT image or a second FFDM image.

18. The method of claim 11, wherein the third set of features is determined using an artificial neural network (ANN) that includes a first encoding module and a second encoding module, the first encoding module configured to determine the first set of features associated with the one or more first medical images or the second set of features associated with the one or more second medical images, the second encoding module configured to determine the third set of features.

19. The method of claim 18, wherein the ANN further includes a decoding module configured to predict the existence or non-existence of the medical abnormality based at least on the third set of features.

20. A non-transitory computer-readable medium comprising instructions that, when executed by a processor included in a computing device, cause the processor to:
obtain one or more first medical images of a person and one or more second medical images of the person, wherein the one or more first medical images are associated with a first mammographic view of the person and wherein the one or more second medical images are associated with a second mammographic view of the person;
determine a first set of features associated with the one or more first medical images;
determine a second set of features associated with the one or more second medical images;
determine a third set of features based on the first set of features and the second set of features, wherein the third set of features represents mammographic characteristics of the person indicated by the first mammographic view and the second mammographic view; and
predict the existence or non-existence of a medical abnormality based at least on the third set of features.
